(19) United States
(12) Patent Application Publication
(10) Pub. No.: US 2020/0377481 A1
(43) Pub. Date: Dec. 3, 2020

(54) SUBSTITUTED 4-(1-PYRROLIDINYL)PYRIMIDINE COMPOUNDS AS DIMERIZATION INHIBITORS OF NEURONAL NITRIC OXIDE SYNTHASE

(71) Applicant: Northwestern University, Evanston, IL (US)

(72) Inventors: Richard B. Silverman, Winnetka, IL (US); Sana Ma, Chicago, IL (US)

(73) Assignee: Northwestern University, Evanston, IL (US)

(21) Appl. No.: 16/307,576

(22) PCT Filed: Jun. 7, 2017
(86) PCT No.: PCT/US2017/036378

§ 371 Date: Dec. 6, 2018

Related U.S. Application Data
(60) Provisional application No. 62/346,939, filed on Jun. 7, 2016.

(51) Int. Cl. C07D 403/14; C07D 405/14; A61P 25/28

(52) U.S. Cl. CPC C07D 403/14; A61P 25/28; C07D 405/14

(57) ABSTRACT

Disclosed are compounds, pharmaceutical compositions comprising the compounds, and methods of using the compounds and pharmaceutical compositions for treating a subject in need thereof. The disclosed compounds may be described as substituted 4-(1-pyrrolidinyl)pyrimidine compounds. The disclosed compounds are shown to inhibit the activity of nitric oxide synthases (NOSs) including neuronal NOS (nNOS) by inhibiting dimerization, and as such, the disclosed compounds and pharmaceutical compositions may be utilized in methods for treating a subject having or at risk for developing a disease or disorder that is associated with nNOS activity.

### CROSS-REFERENCE TO RELATED PATENT APPLICATIONS

This application is the U.S. National Phase under 35 U.S.C. § 371 of International Application No. PCT/US2017/036378, filed on Jun. 7, 2017, which application claims the benefit of priority under 35 U.S.C. § 119(e) to U.S. Provisional Application No. 62/346,939, filed on Jun. 7, 2016, the content of which is incorporated herein by reference in its entirety.

### STATEMENT REGARDING FEDERALLY SPONSORED RESEARCH OR DEVELOPMENT

This invention was made with government support under R01 GM049725 awarded by the National Institutes of Health. The government has certain rights in the invention.

### BACKGROUND

The field of the invention relates to inhibitors of nitric oxide synthases. In particular, the filed of the invention relates to compounds that inhibit the dimerization of neuronal nitric oxide synthase.

The term “neurodegenerative disorder” is used to describe diseases characterized by the progressive breakdown of neuronal function and structure. This term encompasses disorders such as Alzheimer's, Parkinson's, and Huntington's diseases, as well as amyotrophic lateral sclerosis (ALS), among others, although neuronal damage is also associated with stroke and ischemic events, cerebral palsy, and head trauma. Although the human and economic cost of neurodegeneration continues to be astronomical, treatment is largely limited to palliative care and prevention of symptom progression. Therefore, there is a constant demand for novel and effective approaches to slow or prevent the progression of these diseases.

One target under investigation is neuronal nitric oxide synthase (nNOS). Nitric oxide (NO) is an important second messenger in the human body, and dysregulation of its production is implicated in many pathologies. NO is produced by the nitric oxide synthase enzymes, of which there are three isoforms: endothelial nitric oxide synthase (eNOS), which regulates blood pressure and flow, inducible nitric oxide synthase (iNOS), involved in immune system activation, and nNOS, which is required for normal neuronal signaling. Nonetheless, over-expression of nNOS in neural tissue and increased levels of NO can result in protein nitration and oxidative damage to neurons, especially if peroxynitrite is formed from excess NO. Indeed, overexpression of nNOS or excess NO has been implicated in or associated with many neurodegenerative disorders. The inhibition of nNOS is, therefore, a viable therapeutic strategy for preventing or treating neuronal damage.

All NOS enzymes are active only as homodimers. Each monomer consists of both a reductase domain with FAD, FMN, and NADPH binding sites, and a heme-containing oxygenase domain, where the substrate (L-arginine) and cofactor (6R)-5,6,7,8-tetrahydrobiopterin (H_{4B}) bind. Activated and regulated by calmodulin binding, electron flow proceeds from one monomer's reductase domain to the other's oxygenase domain, catalyzing the oxidation of arginine to citrulline with concomitant production of NO. (See, Rosen, G. M.; Tsai, P.; and Pou, S. Mechanism of free-radical generation by nitric oxide synthase. Chem. Rev. 2002, 102 (4), 1191-1199.) Not unexpectedly, most investigated nNOS inhibitors are mimetics of arginine and act as competitive inhibitors.

However, there remains an on-going concern in the art to develop new molecular scaffolds for inhibition of nNOS to benefit patients that suffer from neurological diseases associated with nNOS activity. Here, new inhibitors of nNOS are disclosed that inhibit dimerization.

### SUMMARY

Disclosed are compounds, pharmaceutical compositions comprising the compounds, and methods of using the compounds and pharmaceutical compositions for treating a subject in need thereof. The disclosed compounds are shown to inhibit the activity of nitric oxide synthases (NOSs) including neuronal NOS (nNOS) by inhibiting dimerization, and as such, the disclosed compounds and pharmaceutical compositions may be utilized in methods for treating a subject having or at risk for developing a disease or disorder that is associated with nNOS activity.

The disclosed compounds may be described as substituted 4-(1-pyrrolidinyl)pyrimidine compounds. The disclosed compounds may have a formula described as follows:

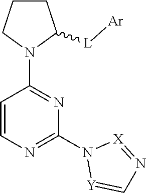

wherein X and Y can be selected from CH and N with the proviso that both of X and Y are not N; L is a divalent alkylene which optionally is substituted, such substituents as can be selected from oxa (—O—) and amido (—C(O)NH— or NHC(O)—) substituents; and Ar can be selected from aryl, heteroaryl and substituted aryl and heteroaryl moieties, such substituents as can be selected from alkyl and alkoxy substituents, such moieties as can be fused to one or more aryl, heteroaryl and non-aromatic cyclo and heterocyclo moieties to form fused rings, and salts thereof

In particular, the disclosed compounds may be described as substituted 2-(1H-imidazole)-4-(1-pyrrolidinyl)pyrimidine compounds. The disclosed compounds may have a formula described as follows:

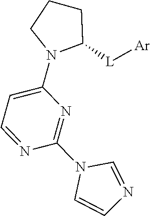

wherein L can be selected from CH₂O(CH₂)ₙ, C(O)NH(CH₂)ₙ and CH₂C(O)NH(CH₂)ₙ moieties, where n can be an integer selected from 1-4 4; and Ar can be selected from substituted and unsubstituted phenyl, indolyl, benzo[d]imidazolyl, benzodioxaolyl, pyridinyl, pyrimidinyl, and pyridazinyl moieties, such substituents as can be selected from methyl, methoxy and divalent methylenedioxy (—OCH₂O—) and ethylenedioxy (—OCH₂CH₂O—) substituents, and salts thereof.

The disclosed compounds may be utilized in various methods. In some embodiments, the disclosed compounds may be utilized in methods for inhibiting, modulating or otherwise affecting dimerization of neuronal nitric oxide synthase. The disclosed methods may be practiced in order to treat and/or prevent diseases or disorders associated with NOS activity.

### BRIEF DESCRIPTION OF THE FIGURES

FIG. 1. Compound PM06.

FIG. 2. Exemplary imidazole derivative compounds.

FIG. 3. Synthesis schemes for intermediates of Compounds 12, 13, and 14.

FIG. 4. Exemplary 1,3-benzodioxole (Compound 12), and dimethoxy-phenyl derivative compounds (Compound 13 and Compound 14).

FIG. 5. Synthesis scheme for amide precursors and indole derivative compounds (Compound SM01027 and Compound SM01028) and benzimidazole derivative compound (Compound SM01025).

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “a component” should be interpreted to mean “one or more components.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Novel compounds are disclosed herein which may be described as substituted 4-(1-pyrrolidinyl)pyrimidine compounds. The disclosed compounds further may be described by various definitions provided herein or known in the art.

The term “alkyl” as used herein refers to a saturated straight or branched hydrocarbon, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of an alkyl group. An exemplary alkylene group is —CH₂— or —CH₂CH₂—.

The term “haloalkyl” refers to an alkyl group that is substituted with at least one halogen. For example, —CH₂F, —CHF_{2,} —CF_{3,} —CH₂CF_{3,} —CF₂CF_{3,} and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number of ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF_{3,} —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The term “amide” or “amido” as used herein refers to a radical of the form —R₁C(O)N(R²)—, R₁C(O)N(R²) R³—, —C(O)N R² R³, or —C(O)NH_{2,} wherein R₁, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated“(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

As another separate consideration, various compounds can be present as an acid salt, either partially or fully protonated. In certain such embodiments, the counter ion can be a conjugate base of a protic acid. Further, it will be understood by those skilled in the art that any one or more of the compounds of this invention can be provided as part of a pharmaceutical composition comprising a pharmaceutically-acceptable carrier component for use in conjunction with a treatment method or medicament.

As relates to certain embodiments, the invention provides a series of analogs that contain a heme-binding moiety, one or more aromatic, or heterocyclic structures containing nitrogen or other structures that bind to iron. The analogs also contain one or more aromatic or heterocyclic moiety that is attached to the heme-binding group via an aliphatic chain. This end moiety has a desired geometry with side groups that interact with surrounding residues. The aliphatic chain has length of approximately 2-6 atoms, with a β-turn configuration. The backbone contains various side-groups in order to create or stabilize this β-turn structure and improve binding of the analog to nNOS. When combined, these moieties are able to successfully inhibit nNOS dimerization.

Without limitation to any one theory or mode of operation, the analogs may be able to inhibit the dimerization of nNOS by binding to the heme group in the oxygenase domain and cause an allosteric effect on the monomer that prevents the monomer to dimerize. Analogs containing variations of an imidazole-pyrimidine substructure are very effective in binding to the heme, but other moieties that are heme-binding can be substituted as well. Attached to the imidazole-pyrimidine moiety is a structure that introduces a β-turn in the molecule, such as D-β homoproline. The homoproline can be homologated at the ester end in a variety of ways to create a substructure of about 2- about 6 atoms long to link an aromatic moiety, such as a dimethoxybenzene or 1,3-benzodioxole, which interacts with propionate residues that enhance the binding and selectivity of the analogs with respect to nNOS monomer.

The disclosed compounds or pharmaceutical compositions may be administered to a subject in need thereof, for example, to treat and/or prevent a disease or disorder associated with NOS activity. The terms “subject,” “patient,” and “individual” may be used interchangeably herein. A subject may be a human subject. A subject may refer to a human subject having or at risk for acquiring a disease or disorder that is associated with nitric oxide synthase (NOS) activity, which may include a disease or disorder that is associated with NOS activity including aberrant NOS. As used herein, the term “aberrant” means higher or lower activity relative to a normal healthy subject. A subject having a disease or disorder associated with nitric oxide synthase activity may include a subject having a disease or disorder associated with neuronal NOS (nNOS), inducible NOS (iNOS), and/or endothelial NOS (eNOS). In specific embodiments, a subject having a disease or disorder associated with nitric oxide synthase activity may include a subject having or at risk for developing a neuronal disease or disorder (e.g., migraine, depression, stroke) and/or a neurodegenerative disease (e.g., Alzheimer's, Parkinson's, and/or Huntington's disease). (See, e.g.,, Mukherjee et al., Chem. Soc. Rev., 2014, Oct. 7; 43(19):6814-6838, the content of which is incorporated herein by reference in its entirety. Inhibitors of NOS that are under clinical development include cindunistat, A-84643, ONO-1714, L-NOARG, NCX-456, VAS-2381, GW-273629, NXN-462, CKD-712, KD-7040, and guanidinoethyldisulfide.

In some embodiments of the disclosed methods, an effective amount of the disclosed compounds or pharmaceutical composition comprising an effective amount of the disclosed compounds may be administered to a subject in need thereof to treat a disease or disorder associated with NOS activity. As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

### ILLUSTRATIVE EMBODIMENTS OF THE DISCLOSED COMPOUNDS AND USES THEREOF

The following embodiments are illustrative and are not intended to limit the scope of the claimed subject matter.

Embodiment 1. A compound or a salt thereof having a formula:

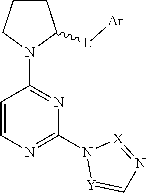

wherein: X and Y are selected from CH and N with the proviso that both of X and Y are not N; L is a divalent alkylene moiety optionally substituted with substituents selected from —O—, _13 C(O)NH—, and —NHC(O)—; and Ar is selected from aryl, heteroaryl and substituted aryl and heteroaryl moieties, such substituents selected from alkyl and alkoxy substituents.

Embodiment 2. The compound of embodiment 1, wherein both of X and Y are CH.

Embodiment 3. The compound of embodiment 1 or 2, wherein L has a formula —(CH₂)ₘ—Z—(CH₂)ₙ—; m is selected from 0-4, n is selected from 0-4, and Z is selected from —CH₂—, —O—, —C(O)NH—, and —NHC(O)—.

Embodiment 4. The compound of any of the foregoing embodiments, wherein Ar is a saturated or unsaturated carbon homocycle or heterocycle group comprising one 5- or 6-membered ring or comprising two or three fused 5- or 6-membered rings optionally substituted with alkyl or alkyoxy substituents.

Embodiment 5. The compound of any of the foregoing embodiments, wherein Ar is selected from phenyl, 1,3-benzodioxole, indole, benzimidazole, pyridine, pyrimidine, and pyridazine.

Embodiment 6. The compound of any of the foregoing embodiments having a formula selected from:

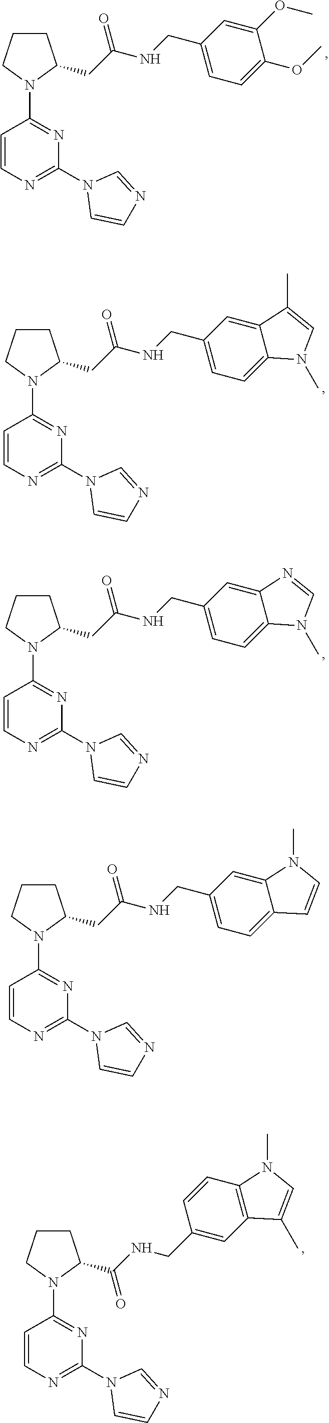 [SMILES: COc1ccc(CNC(=O)C[C@H]2CCCN2c2ccnc(-n3ccnc3)n2)cc1OC ; Cc1cn(C)c2ccc(CNC(=O)C[C@H]3CCCN3c3ccnc(-n4ccnc4)n3)cc12 ; Cn1cnc2cc(CNC(=O)C[C@H]3CCCN3c3ccnc(-n4ccnc4)n3)ccc21 ; Cn1ccc2ccc(CNC(=O)C[C@H]3CCCN3c3ccnc(-n4ccnc4)n3)cc21 ; Cc1cn(C)c2ccc(CNC(=O)[C@H]3CCCN3c3ccnc(-n4ccnc4)n3)cc12]

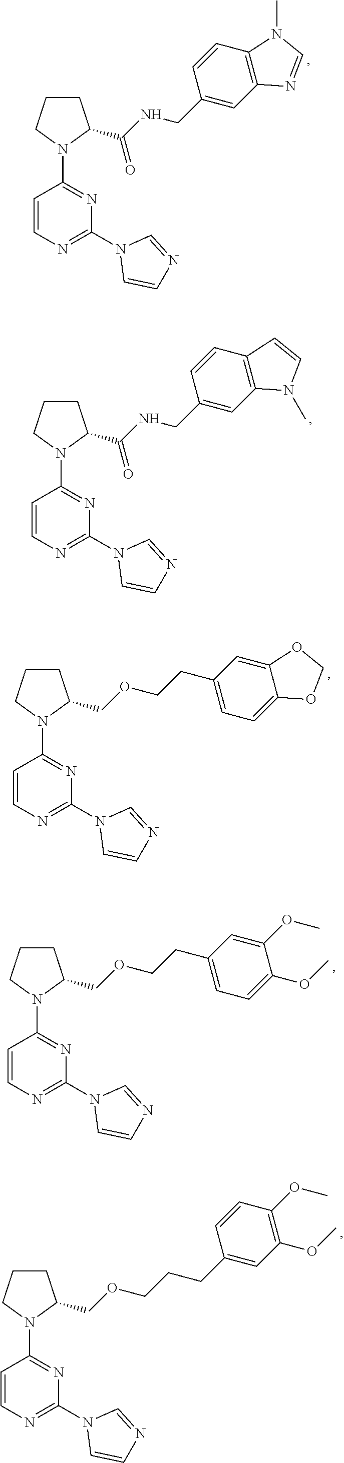 [SMILES: Cn1cnc2cc(CNC(=O)[C@H]3CCCN3c3ccnc(-n4ccnc4)n3)ccc21 ; Cn1ccc2ccc(CNC(=O)[C@H]3CCCN3c3ccnc(-n4ccnc4)n3)cc21 ; c1cn(-c2nccc(N3CCC[C@@H]3COCCc3ccc4c(c3)OCO4)n2)cn1 ; COc1ccc(CCOC[C@H]2CCCN2c2ccnc(-n3ccnc3)n2)cc1OC ; COc1ccc(CCCOC[C@H]2CCCN2c2ccnc(-n3ccnc3)n2)cc1OC]

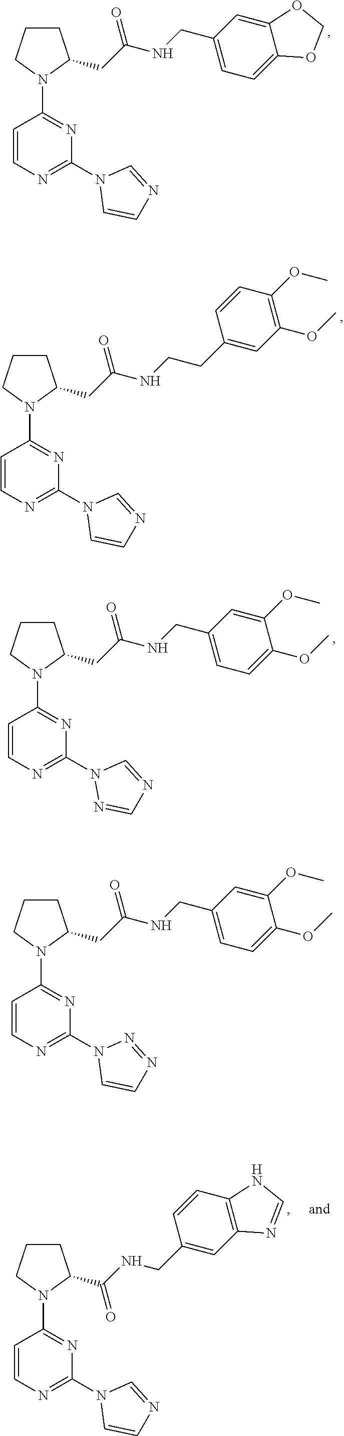 [SMILES: O=C(C[C@H]1CCCN1c1ccnc(-n2ccnc2)n1)NCc1ccc2c(c1)OCO2 ; COc1ccc(CCNC(=O)C[C@H]2CCCN2c2ccnc(-n3ccnc3)n2)cc1OC ; COc1ccc(CNC(=O)C[C@H]2CCCN2c2ccnc(-n3cncn3)n2)cc1OC ; COc1ccc(CNC(=O)C[C@H]2CCCN2c2ccnc(-n3ccnn3)n2)cc1OC ; O=C(NCc1ccc2[nH]cnc2c1)[C@H]1CCCN1c1ccnc(-n2ccnc2)n1]

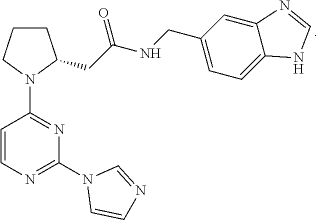 [SMILES: O=C(C[C@H]1CCCN1c1ccnc(-n2ccnc2)n1)NCc1ccc2[nH]cnc2c1]

Embodiment 7. A pharmaceutical composition comprising the compound of any of the foregoing embodiments and a suitable carrier.

Embodiment 8. The compound or composition of any of the foregoing embodiments for use in treating a disease or disorder associated with neuronal nitric oxide synthase activity in a subject in need thereof.

Embodiment 9. The compound or composition of any of the foregoing embodiments, wherein the disease or disorder is neuropathic pain associated with neuronal nitric oxide synthase activity.

Embodiment 10. The compound or composition of any of the foregoing embodiments, wherein the disease or disorder is a neurodegenerative disease.

Embodiment 11. The compound or composition of any of the foregoing embodiments, wherein the neurodegenerative disease is Alzheimer's disease.

Embodiment 12. The compound or composition of any of the foregoing embodiments, wherein the neurodegenerative disease is Parkinson's disease.

### Synthesis Methods

Using imidazolyl, pyrimidyl, prolinyl, linker and aromatic substructures of the sort described above, compound PM106 (compound 2 in Scheme 1, below) and substituent variations thereof can be prepared as shown in Scheme 1. (Compounds 1-5 are characterized by spectral data, as provided in Examples 1a-e below.)

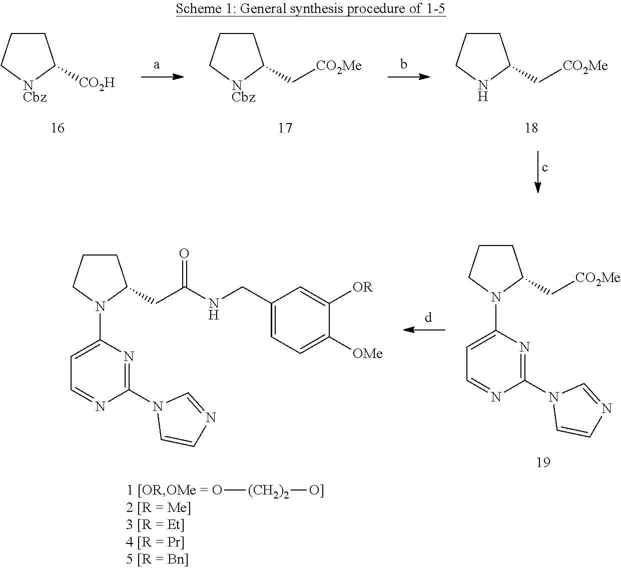

Reagents and conditions: (a) (i) oxalyl chloride, cat. DMF, CH₂Cl_{2,} RT, 5 h, (ii) TMSCHN_{2,} triethylamine, THF/MeCN (1:1), 0° C., 12 h, 86%, (iii) AgOBz, MeOH, 60° C., 6 h, 78%; (b) Pd/C, H_{2,} MeOH, RT, 1 h, 96%; (c) (i) 4-chloro-2-methanesulfonyl pyrimidine, K₂CO_{3,} MeCN, 40° C., 12 h, then imidazole, 65° C., 36 h, 76%; (d) (i) LiOH, THF/H₂O, RT, 12 h, 79%, (ii) Amine, carbonyldiimidazole, DMF, RT, 5 h.

TABLE 1
Biological data.

| | IC₅₀ (μM) | | |
|---|---|---|---|
| compd | nNOS | eNOS | iNOS |
| 1 | 0.20 | 18 | 0.1 |
| 2 | 0.14 | 21.1 | 0.62 |
| 3 | 6.7 | 57 | 3.7 |
| 4 | 7.4 | 86 | 4.5 |
| 5 | 14.1 | 55 | 6.0 |

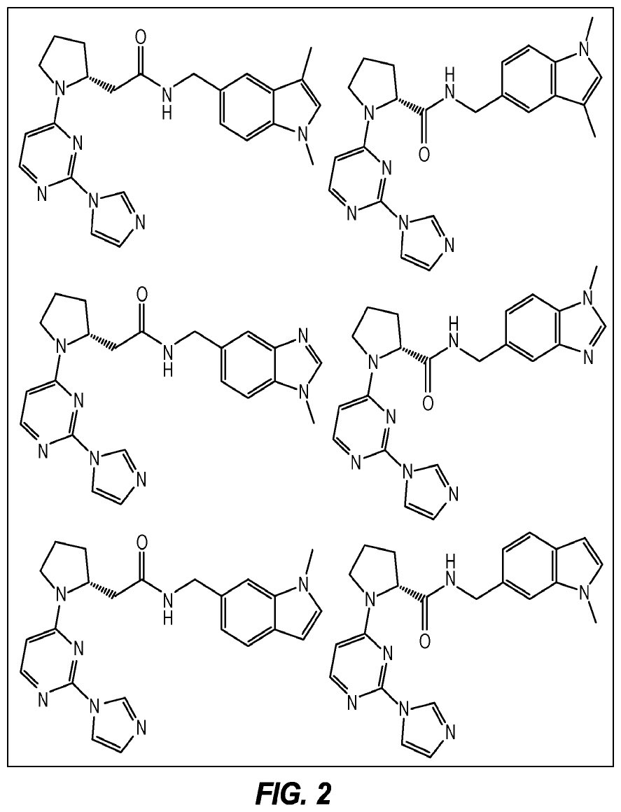

Various other aryl and nitrogenous heteroaryl substructures can be used in conjunction with the compounds of this invention, such substructures as would be understood by those skilled in the art and made aware of this invention. For instance, one or both of the imidazolyl and pyrimidyl moieties of the present compounds can be replaced with 5- and 6- membered aryl and heteroaryl moieties of the sort described in co-pending application Ser. No. 14/798,307 filed on Jul. 13, 2015, published as U.S. 2016/0009690, the entirety of which is incorporated herein by reference. Without limitation, the imidazolyl-pyrimidyl substructure of the compounds of this invention can be replaced with a substructure of a formula

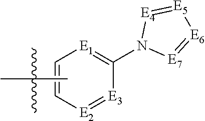

wherein E₁-E₃ can be independently selected from CH and N; and E₄-E₇ can be independently selected from CH, CR₁ and N, providing at least one of E₄-E₇ is N, and where R₁ can be selected from methyl and halo substituents. Without limitation, at least one of E₁-E₃ can be N. In certain embodiments, E₁ and E₃ can be N, and E₅ can be N. (See, e.g., the aforementioned ‘307 application at paragraphs [0009] and [0011], and representative substructures illustrated in FIGS. 2 and 4-6 thereof) Regardless, such substructures can be prepared from reaction between corresponding pyrimidine and imidazole starting materials or analogs thereof using synthetic techniques of the sort described below or straightforward modifications thereof, such modifications as would also understood by those skilled in the art. Such replacement substructures and/or moieties are limited only by ability to bind, complex or otherwise functionally interact with the heme-iron group of the oxygenase domain of nNOS and adversely affect or modulate dimerization.

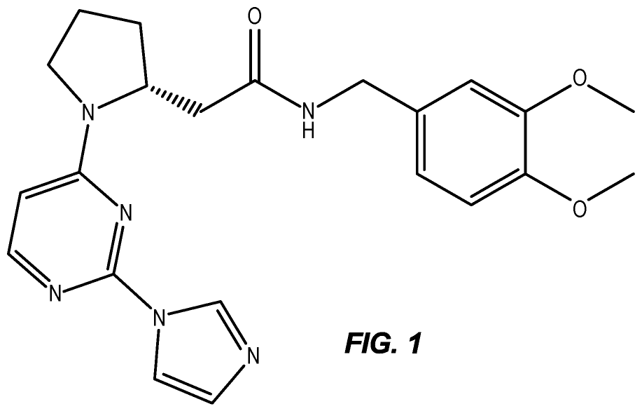

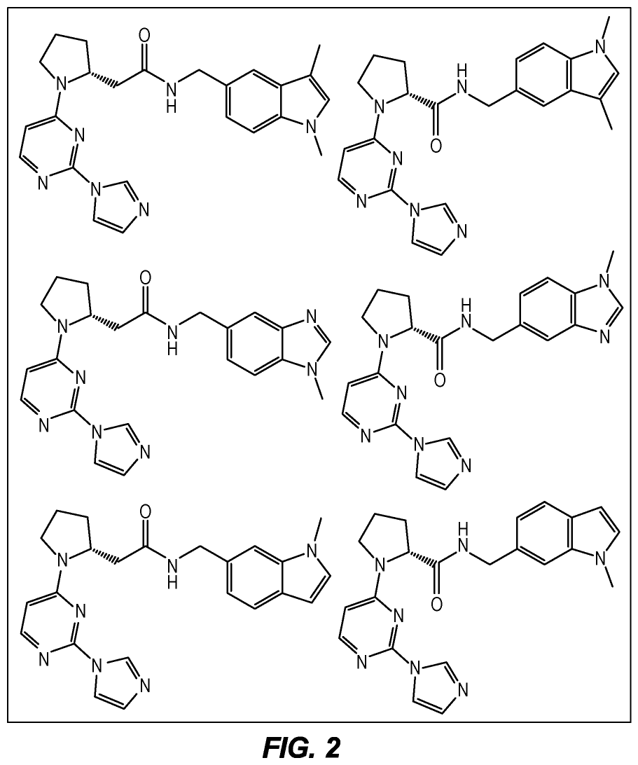

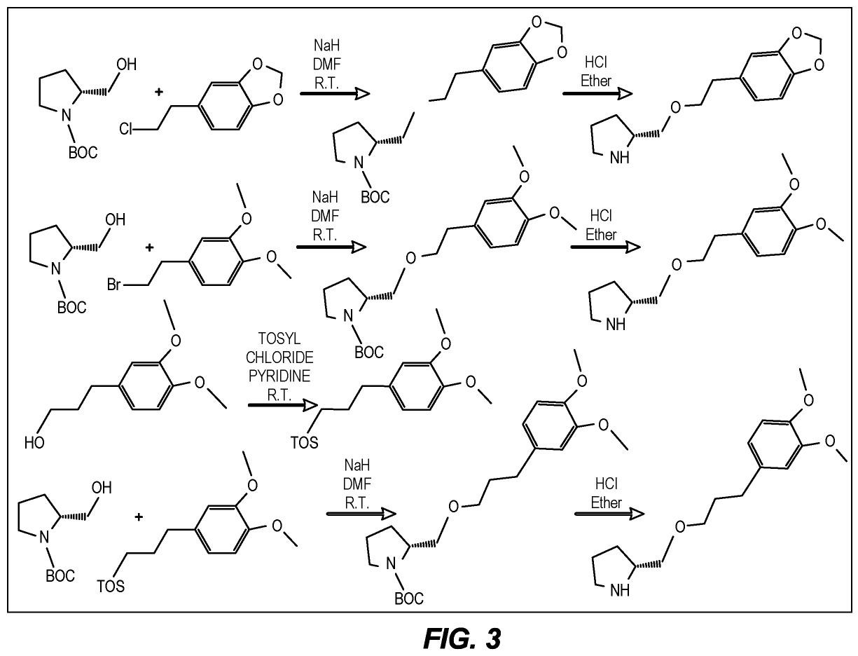

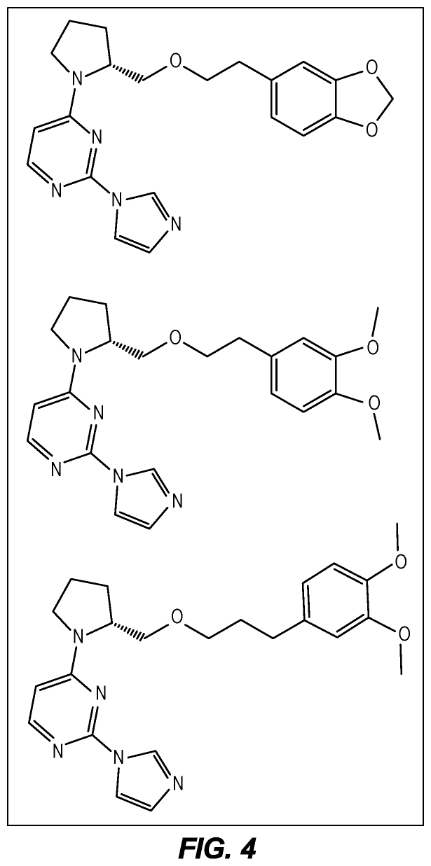

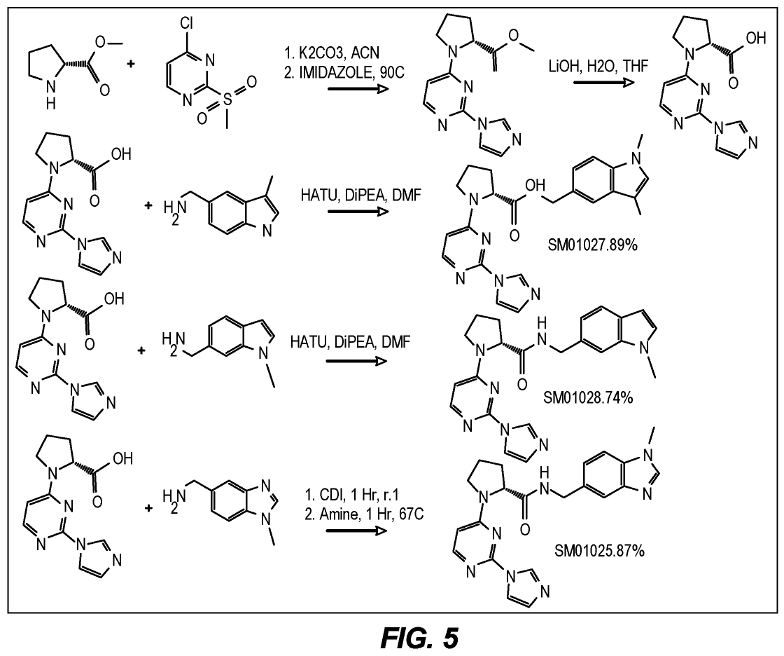

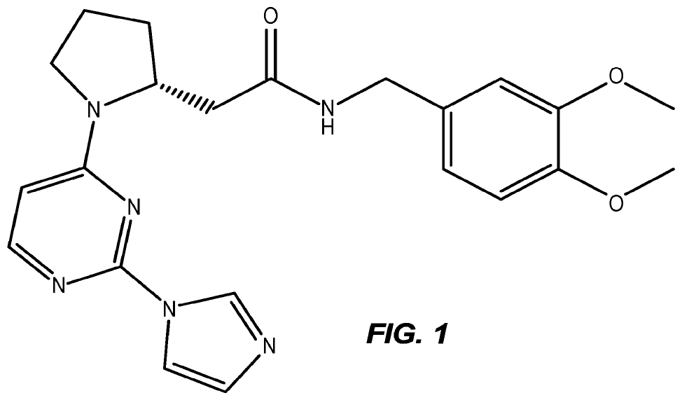

Several representative terminal aromatic substructures useful in conjunction with the compounds of this invention are illustrated by way of Examples 2-5 and FIGS. 1, 2, and 4. Various other aromatic substructures can be incorporated therein, as would be understood by those skilled in the art and made aware of this invention. For instance, such substructures can be replaced with appropriately substituted 5- and 6- numbered aryl and heteroaryl moieties, such moieties as can be fused to one or more aryl, heteroaryl and non-aromatic cyclo and heterocyclo moieties. Such replacement substructures, moieties and/or substituents are limited only by ability to bind, complex or otherwise functionally interact with propionate and other such residues to enhance interaction of the compounds of this invention with an nNOS monomer and adversely affect or modulate dimerization.

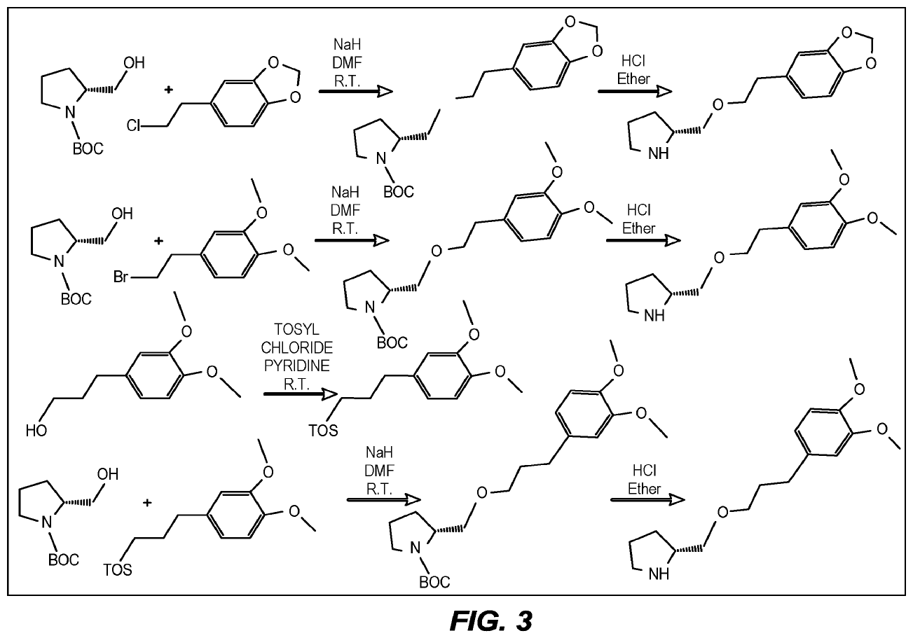

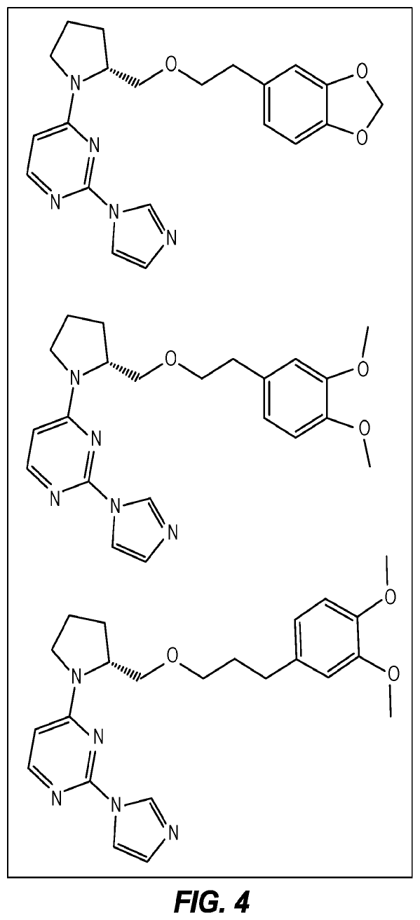

While amido substructures are shown in conjunction with compounds 1-5, to link the aforementioned terminal substructures, variations thereof can be employed through choice of prolinyl and terminal aromatic substructures. (See e.g., Example 10 and FIGS. 3 and 4.) For instance, an amido linker can be replaced with a range of ether substructures, as shown in FIG. 3, through choice of prolinyl and terminal aromatic starting materials. Such replacement substructures are limited only by way of function to provide and/or stabilize a β-turn configuration within the compounds of this invention, improve binding thereof to an nNOS monomer and adversely affect or modulate dimerization.

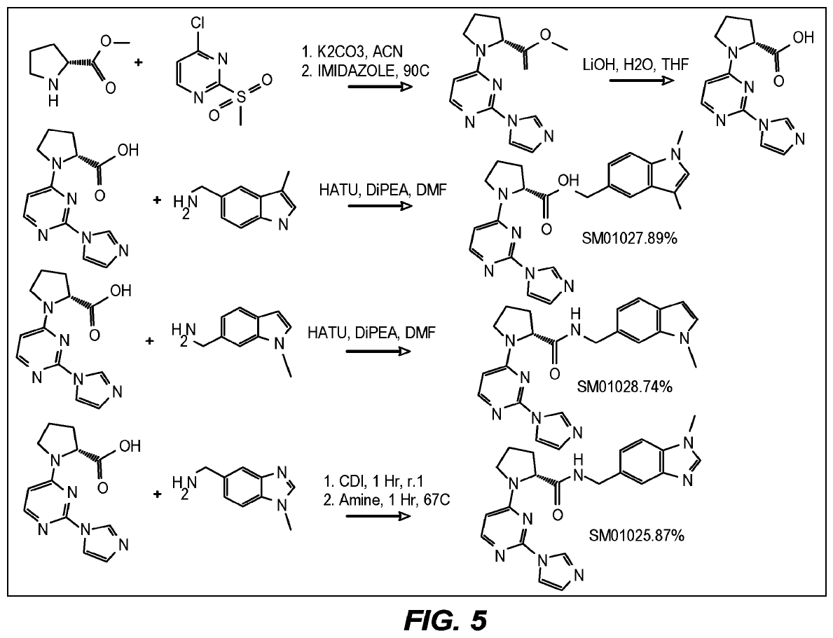

Compounds of the present invention can be prepared using such substructures in accordance with the synthetic procedures outlined in Schemes 1 and 3, Example 6 and FIG. 5 or straight-forward modifications thereof, such modifications and resulting compounds limited only by commercial or synthetic availability of corresponding starting materials (e.g., chloro-substituted nitrogenous heteroaryl and alkylamine aromatic materials) and reagents.

Methods of the present invention can also, as would be understood by those skilled in the art, be extended to or include methods using or in conjunction with a pharmaceutical composition comprising an inhibitor compound of the sort described herein and a physiologically or otherwise suitable formulation. In a some embodiments, the present invention includes one or more NOS inhibitors, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable sterile aqueous or nonaqueous solutions, dispersions, suspensions or emulsions. The resulting compositions can be, in conjunction with the various methods described herein, for administration or contact with a cellular medium, bacterium and/or a nitric oxide synthase expressed or otherwise present therein. Whether or not in conjunction with a pharmaceutical composition, “contacting” means that a nitric oxide synthase and one or more inhibitor compounds are brought together for purpose of binding and/or complexing such an inhibitor compound to the enzyme. Amounts of a compound effective to inhibit a nitric oxide synthase may be determined empirically, and making such determinations is within the skill in the art. Modulation, inhibition or otherwise affecting nitric oxide synthase activity includes both reduction and/or mitigation, as well as elimination of NOS activity and/or nitric oxide production.

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically-acceptable salt thereof, or related composition may be administered in two or more sub-doses, administered separately over an appropriate period of time.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, Pa.

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the present invention provides for use of one or more nitric oxide synthase inhibitor compounds for the manufacture of a medicament for therapeutic use in the treatment of various disease states, in particular neurodegenerative diseases and neuropathic pain.

### EXAMPLES

The following Examples are illustrative and are not intended to limit the scope of the claimed subject matter.

### Example 1

The following non-limiting examples and data illustrate various aspects and features relating to the compounds and methods of the present invention. In comparison with the prior art, the present methods provide results and data which are surprising, unexpected and contrary thereto. While the utility of this invention is illustrated through the use of several nNOS inhibitor compounds, and moieties thereof, it will be understood by those skilled in the art that comparable results are obtainable with various other nNOS inhibitor compounds and/or moieties, as are commensurate with the scope of this invention.

### Example 1a

(R)-2-[2-(1H-imidazol-1-yl)-4-pyrimidylpyrrolidin-2-yl)-N-(benzo[d][1,3]dioxo-5-ylmethyl)acetamide (1). Synthesis performed using benzo[d][1,3]dioxol-5-ylmethylamine with general procedure Scheme 1. ¹H NMR (500 MHz; CDCl₃): δ 8.52 (s, 1 H), 8.11 (d, J=6.0 Hz, 1 H), 7.79 (s, 1 H), 7.08 (s, 1 H), 6.74 (d, J=7.9 Hz, 1 H), 6.73-6.66 (m, 2 H), [6.39 (br s), 6.17 (br s), 1:4, 1 H], 5.94 (s, 2 H), 5.77 (s, 1 H), 4.73 (s, 1 H), 4.37 (dd, J=14.3, 5.6 Hz, 1 H), 4.28 (d, J=11.7 Hz, 1 H), [3.71 (br s), 3.46 (br s), 1:4, 1 H], [3.64 (br s), 3.32 (br s), 1:4, 1 H], [2.82 (s), 2.58 (s), 4:1, 1 H], 2.35 (s, 1 H), 2.15-2.07 (m, 4 H). ¹³C NMR (126 MHz; CDCl₃): δ 170.0, 160.4, 156.1, 154.1, 147.9, 147.1, 136.1, 131.8, 130.1, 121.2, 116.5, 108.3, 101.8, 101.1, 55.4, 47.0, 43.5, 39.6, 30.5, 23.3.

### Example 1b

(R)-2-[2-(1H-imidazol-1-yl)-4-pyrimidylpyrrolidin-2-yl]-N-(3,4-dimethoxybenzyl) acetamide (2). Synthesis performed using 3,4-dimethoxy benzylamine with general procedure Scheme 1. ¹HNMR (500 MHz; CDCl₃): δ 8.53 (s, 1 H), 8.11 (d, J=6.0 Hz, 1 H), 7.80 (s, 1 H), 7.08 (s, 1 H), 6.81-6.78 (m, 3 H), [6.42 (s), 6.15 (s), 1:4, 1 H], 5.77 (s, 1 H), 4.74 (s, 1 H), 4.42 (dd, J=14.3, 5.9 Hz, 1 H), 4.30 (dd, J=14.3, 5.3 Hz, 1 H), 3.86 (s, 3 H), 3.85 (s, 3 H), 3.44 (s, 1 H), 3.32 (s, 1 H), [2.83 (s), 2.59 (s), 4:1, 1 H], 2.41-2.31 (m, 1 H), 2.25-1.90 (m, 4 H). ¹³C NMR (126 MHz; CDCl₃): δ 170.1, 160.3, 156.1, 154.1, 149.2, 148.6, 136.1, 130.6, 130.1, 120.2, 116.5, 111.3, 111.2, 101.8, 56.0, 55.5, 47.0, 43.6, 39.7, 30.5, 23.3.

### Example 1c

(R)-2-[2-(1H-imidazol-1-yl)-4-pyrimidylpyrrolidin-2-yl]-N-(3-ethoxy-4-methoxybenzy)acetamide (3). Synthesis performed using 3-ethoxy-4-methoxybenzylamine with general procedure Scheme 1. ¹H NMR (500 MHz; CDCl₃): δ 8.53 (s, 1 H), 8.11 (d, J=6.0 Hz, 1 H), 7.80 (s, 1 H), 7.08 (s, 1 H), 6.91-6.67 (m, 3 H), [6.42 (s), 6.15 (s), 1:5, 1 H], 5.73 (s, 1 H), 4.74 (s, 1 H), 4.40 (dd, J=14.3, 5.8 Hz, 1 H), 4.28 (dd, J=14.3, 5.3 Hz, 1 H), 4.06 (q, J=7.0 Hz, 2 H), 3.85 (s, 3 H), 3.44-3.31 (m, 2 H), [2.85 (s), 2.60 (s), 5:1, 1 H], 2.32 (s, 1 H), 2.24-1.92 (m, 4 H), 1.45 (t, J=7.0 Hz, 3 H). ¹³C NMR (126 MHz; CDCl₃): δ 170.1, 160.3, 156.1, 154.0, 148.9, 148.5, 136.1, 130.6, 130.1, 120.2, 116.5, 112.7, 111.5, 101.8, 64.4, 56.0, 55.5, 47.0, 43.6, 39.6, 30.5, 23.3, 14.8.

### Example 1d

(R)-2-[2-(1H-imidazol-1-yl)-4-pyrimidylpyrrolidin-2-yl]-N-(4-methoxy-3-n-propylbenzyl)acetamide (4). Synthesis performed using 4-methoxy-3-n-propylbenzylamine with general procedure Scheme 1. ¹H NMR (500 MHz; CDCl₃): δ 8.53 (s, 1 H), 8.11 (d, J=6.0 Hz, 1 H), 7.80 (s, 1 H), 7.08 (s, 1 H), 6.86-6.70 (m, 3 H), [6.42 (s), 6.15 (s), 1:5, 1 H], 5.73 (s, 1 H), 4.74 (s, 1 H), 4.41 (dd, J=14.3, 5.9 Hz, 1 H), 4.27 (dd, J=14.1, 4.8 Hz, 1 H), 3.94 (t, J=6.8 Hz, 2 H), 3.85 (s, 3 H), 3.58-3.31 (m, 2 H), [2.83 (s), 2.55 (s), 5:1, 1 H], 2.33 (s, 1 H), 2.14-2.06 (m, 4 H), 1.85 (h, J=7.3 Hz, 2 H), 1.03 (t, J =7.4 Hz, 3 H). ¹³C NMR (126 MHz; CDCl₃): δ 170.0, 160.3, 156.1, 154.1, 149.0, 148.8, 136.1, 130.6, 130.1, 120.2, 116.5, 113.0, 111.8, 101.8, 70.6, 56.1, 55.5, 47.0, 43.6, 39.7, 30.5, 23.3, 22.5, 10.5.

### Example 1e

(R)-2-[2-(1H-imidazol-1-yl)-4-pyrimidylpyrrolidin-2-yl]-N-(3-benzyl-4-methoxybenzyl)acetamide (5). Synthesis performed using 3-benzyl-4-methoxy benzylamine with general procedure Scheme 1. ¹H NMR (500 MHz; CDCl₃): δ 8.53 (s, 1 H), 8.11 (d, J=6.0 Hz, 1 H), 7.78 (s, 1 H), 7.42 (d, J=7.3 Hz, 2 H), 7.34 (t, J=7.5 Hz, 2 H), 7.28 (d, J=7.6 Hz, 1 H), 7.07 (s, 1 H), 6.84-6.77 (m, 3 H), [6.39 (s), 6.15 (s), 1:4, 1 H], 5.67 (t, J=5.0 Hz, 1 H), 5.11 (s, 2 H), 4.70 (s, 1 H), 4.36 (dd, J=14.4, 5.8 Hz, 1 H), 4.25 (dd, J=14.4, 5.3 Hz, 1 H), 3.87 (s, 3 H), 3.49-3.28 (m, 2 H), [2.81 (s), 2.55 (s), 4:1, 1 H] 2.30 (s, 1 H), 2.17-1.97 (m, 4 H). ¹³C NMR (126 MHz; CDCl₃): δ 170.0, 160.3, 156.1, 154.0, 149.3, 148.3, 136.9, 136.1, 130.5, 130.1, 128.5, 128.1, 127.8, 127.3, 120.8, 116.5, 114.0, 111.9, 101.8, 71.0, 56.1, 55.5, 47.0, 43.5, 39.6, 30.5, 23.2.

### Example 2

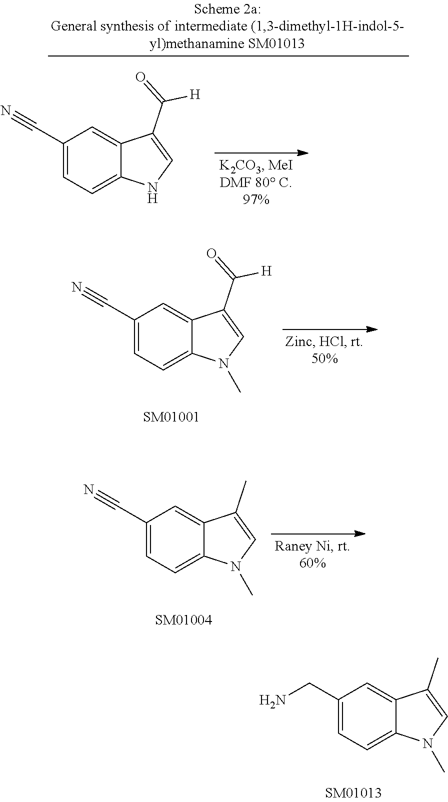

### Example 2a

Step 1: 3-formyl-1H-indole-5-carbonitrile, 1 g (1 eq, 5.9 mmol) was added to 50 mL round bottom flask, along with 1.63 g of potassium carbonate (2 eq, 11.8 mmol), dissolved in 30 mL of DMF. Mixture stirred at 80C. 0.81 mL of iodomethane (2.2 eq, 12.98 mmol) was added into the flask, let stirred for 24 hours. Residual solvent evaporated. Product crystallized with ethyl acetate, then filtered out. yield 99%. ¹H NMR (500 MHz, DMSO-d₆) δ 9.98 (s, 2H), 8.52-8.46 (m, 4H), 7.83 (d, J=8.5 Hz, 2H), 7.74 (dd, J=8.5, 1.6 Hz, 2H), 3.96 (s, 6H).

### Example 2b

Step 2: 3-formyl-1-methyl-1H-indole-5-carbonitrile, 200 mg (1 eq,) was added to a round bottom flask, dissolved in 10 mL THF, stirring in ice bath, 0C. Zinc powder was added to the mixture (1.1 eq). A drop of HCl concentrated was added. Mixture stirred for 8 hours at room temperature. Water was added, then THF was evaporated until water remained, and product extracted with ethyl acetate and water. organic layer purified with silica column chromatography with DCM and ethyl acetate solvent. yield, 50%. ¹H NMR (500 MHz, DMSO-d₆) δ 8.05 (d, J=1.5 Hz, 3H), 7.58 (d, J=8.5 Hz, 3H), 7.48 (dd, J=8.5, 1.5 Hz, 3H), 7.30 (d, J=1.2 Hz, 3H), 3.78 (s, 8H), 2.27 (d, J=1.0 Hz, 8H).

### Example 2c

Step 3: Raney nickel in water was washed with methanol, then added to 50 mL round bottom flask with ammonia 7N in methanol. 1,3-dimethyl-1H-indole-5-carbonitrile, 250 mg (1 eq.) was added to flask. Reaction let stirring under hydrogen balloon pressure at 50C for 24 hours. Final mixture was centrifuged to collect salt as a pellet. Remaining fluid was evaporated to remove residual solvent. Final purification with reverse phase C18 column chromatography with water and acetonitrile. Yield, 64% NMR (500 MHz, Chloroform-d) δ 7.47 (s, 1H), 7.22 (d, J=8.4 Hz, 2H), 7.15 (dd, J=8.5, 1.6 Hz, 1H), 6.80 (s, 1H), 3.95 (s, 2H), 3.70 (s, 3H), 2.39-2.13 (m, 3H).

### Example 3

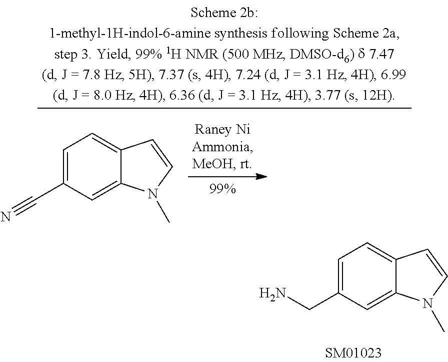

### Examples 4 and 5

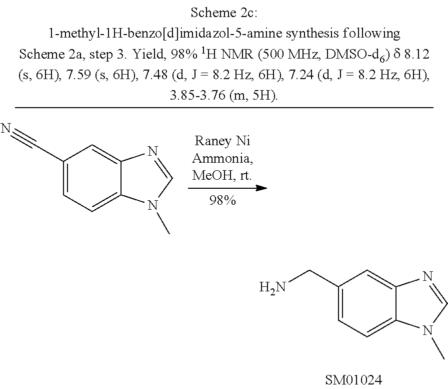

### Example 6

With reference to FIG. 2, compounds 6-11 can be prepared in accordance with representative syntheses outlined in Scheme 3 and as further detailed in Examples 6d-i, below. (These and other related indolyl and benzo[d]imidazolyl compounds and associated amido linker substructures, prepared in accordance with the procedures described herein, are provided in FIG. 5.)

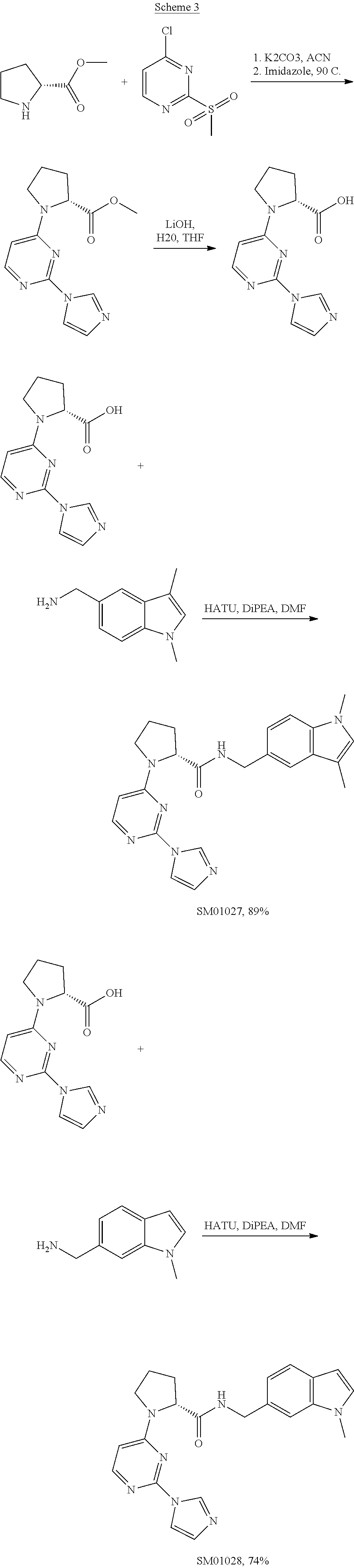

### Example 6a

Step 1: 1.64 g (1 eq, 9.9 mmol) of H-D-Proline-OMe was dissolved with 2-3 mL DCM in a dried round bottom flask. 4-chloro-2-(methylsulfonyl)pyrimidine (1.1 eq, 10.95 mmol) was then added into the reaction beaker. 75 mL of acetonitrile was then added, along with 7.5g potassium carbonate (3 eq, 54.33 mmol). The entire mixture was stirred at 40C for 3 days. Then, 0.74 g of imidazole (1.1 eq, 10.89 mmol) was added to the same reaction flask, and temperature raised to 90C, stirred for another 3 days. Another 10 mL of DMF was added to the reaction flask to aid reaction completion. Residual solvent was evaporated under high vacuum, then product was taken up with ethyl acetate and filtered to remove salts. Final purification with silica column chromatography, DCM and methanol solvents, 93%.

### Example 6b

Step 2: 2 g of methoxy imidazole (1 eq, 7.326 mmol) was added to 500 mL round bottom flask and dissolved in THF. Lithium hydroxide and water was then added to the flask, stirred for 24 hours on ice bath (0C). Final mixture was purified with ion exchange chromatography, then crystallized with methanol and ethyl acetate, 53%.

### Example 6c

Step 3: carboxylic acid, 50 mg (1 eq, 0.1928 mmol) was added to 50 mL round bottom flask, dissolved with DMF. DiPEA, 0.1 mL (3 eq, 0.5784 mmol) was then added to the flask, followed by HATU, 73.30 mg (1 eq, 0.1928). Amine (e.g., an indolylmethaneamine from Examples 2 or 3) was added, 31.08 mg (1 eq, 0.1928 mmol). Mixture was stirred for 2 days at room temperature. Product extracted with ethyl acetate and water, then purified with column chromatography with DCM and methanol, then with prep-HPLC.

### Example 6d

(R)-2-(1-(2-(1H-imidazol-1-yl)pyrimidin-4-yl)pyrrolidin-2-yl)-N-((1,3-dimethyl-1H-indol-5-yl)methyl)acetamide (6) Reaction performed using Scheme 1, with (1,3-dimethyl-1H-indol-5-yl)methanamine. ¹H NMR (500 MHz, Chloroform-d) δ 8.65 (s, 1H), 7.79 (s, 1H), 7.38 (s, 1H), 7.16 (dd, J=8.4, 1.5 Hz, 1H), 7.05 (dd, J=8.4, 1.7 Hz, 1H), 7.00-6.95 (m, 1H), 6.79 (d, J=1.5 Hz, 1H), 6.14-6.09 (m, 1H), 5.99 (s, 1H), 4.73 (s, 1H), 4.48 (qd, J=14.1, 5.1 Hz, 2H), 3.68 (d, J=1.5 Hz, 3H), 3.42 (s, 1H), 3.26 (s, 1H), 2.78 (s, 1H), 2.25 (d, J=1.2 Hz, 3H), 2.10 (s, 2H), 2.06-2.02 (m, 3H). yield, 64%

### Example 6e

(R)-2-(1-(2-(1H-imidazol-1-yl)pyrimidin-4-yl)pyrrolidin-2-yl)-N-((1-methyl-1H-benzo[d]imidazol-5-yl)methyl)acetamide (7) Reaction will be performed using Scheme 1, with 1-methyl-1H-benzo[d]imidazol-5-amine.

### Example 6f

(R)-2-(1-(2-(1H-imidazol-1-yl)pyrimidin-4-yl)pyrrolidin-2-yl)-N-((1-methyl-1H-indol-6-yl)methyl)acetamide (7) Reaction performed using Scheme 1, with 1-methyl-1H-indol-6amine. ¹H NMR (500 MHz, Chloroform-d) δ 8.60 (s, 1H), 8.10 (d, J=6.0 Hz, 1H), 7.83 (s, 2H), 7.59 (d, J=8.1 Hz, 1H), 7.24 (s, 1H), 7.08 (d, J=3.1 Hz, 2H), 7.02 (d, J=8.1 Hz, 2H), 6.49 (dd, J=3.1, 0.8 Hz, 2H), 6.15 (s, 1H), 4.78 (s, 1H), 4.65-4.49 (m, 3H), 3.79 (s, 4H), 3.46 (s, 1H), 3.31 (s, 1H), 3.01 (s, 1H), 2.35 (s, 1H), 2.17 (s, 2H). yield, 89%.

### Example 6g

(R)-1-(2-(1H-imidazol-1-yl)pyrimidin-4-yl)-N-((1,3-dimethyl-1H-indol-5-yl)methyl)ppyrrolidine-2-carboxamide (9) Reaction performed using Scheme 3, with (1,3-dimethyl-1H-indol-5-yl)methanamine. Amine was prepared using Scheme 2 ¹H NMR (500 MHz, Chloroform-d) δ 8.68 (s, 0H), 8.10 (d, J=5.9 Hz, 1H), 7.62-7.58 (m, 1H), 7.31-7.23 (m, 1H), 7.03-6.94 (m, 2H), 6.73 (d, J=4.9 Hz, 1H), 6.25 (s, 1H), 4.74 (s, OH), 4.49 (t, J=5.2 Hz, 1H), 4.09 (q, J=7.1 Hz, 1H), 3.63 (d, J=4.5 Hz, 2H), 3.49 (s, 1H), 3.40 (s, 1H), 2.35-2.28 (m, 1H), 2.22 (s, 1H), 2.06 (s, 1H), 2.01 (s, 2H), 1.23 (t, J=7.1 Hz, 2H). Yield, 89%.

### Example 6h

(R)-1-(2-(1H-imidazol-1-yl)pyrimidin-4-yl)-N-((1-methyl-1H-benzo[d]imidazol-5-yl)methyl)pyrrolidine-2-carboxamide (10) Reaction will be performed using Scheme 3, with 1-methyl-1H-benzo[d]imidazol-5-amine.

### Example 6i

(R)-1-(2-(1H-imidazol-1-yl)pyrimidin-4-yl)-N-((1-methyl-1H-indol-6-yl)methyl)pyrrolidine-2-carboxamide (11) Reaction performed using Scheme 3, with 1-methyl-1H-indol-6-amine. ¹H NMR (500 MHz, Chloroform-d) δ 8.58 (s, 1H), 8.11 (d, J=5.9 Hz, 1H), 7.62 (s, 1H), 7.40-7.32 (m, 1H), 7.07 (d, J=4.0 Hz, 1H), 6.96 (d, J=3.7 Hz, 1H), 6.87 (t, J=8.1 Hz, 2H), 6.81 (s, 1H), 6.37 (d, J=6.5 Hz, 1H), 6.25 (s, 1H), 4.69 (s, 1H), 4.51 (d, J=5.9 Hz, 2H), 3.66-3.55 (m, 5H), 3.40 (s, 1H), 3.14 (s, 2H), 2.32 (ddd, J=10.0, 6.4, 3.4 Hz, 1H), 2.21 (s, 2H). Yield, 74%.

### Example 7

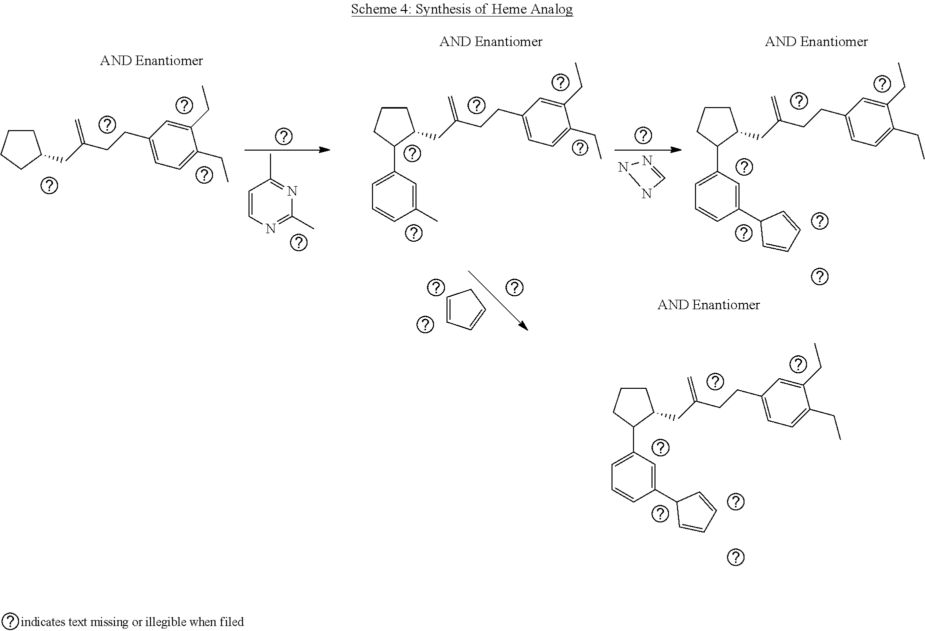

### Example 8

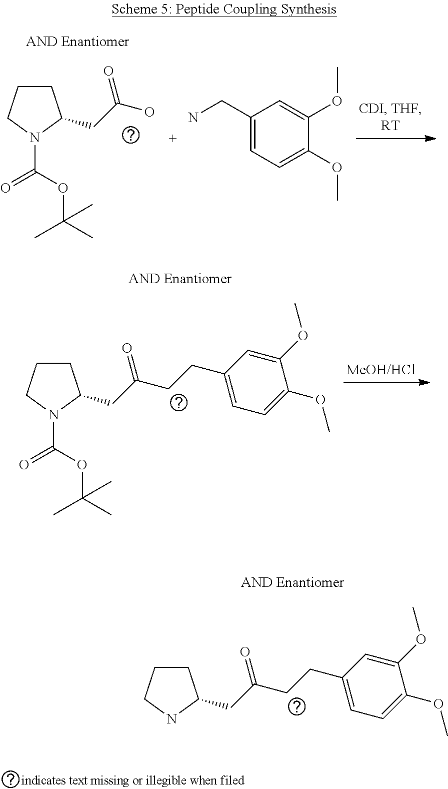

### Example 9

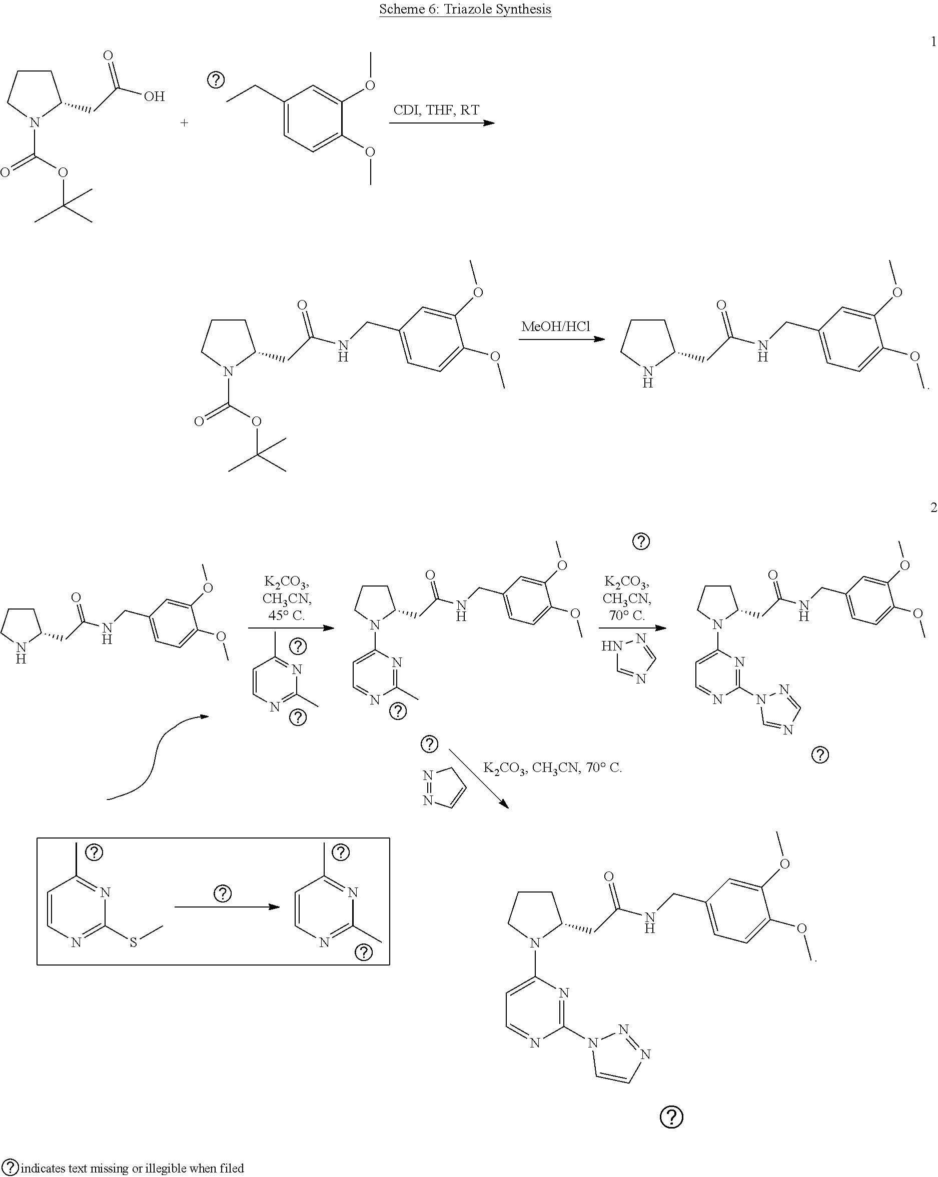

### Example 10

The amido linkages of compounds illustrated in the foregoing examples can be replaced with ether linker substructures. For instance, a halogenated benzodioxole or dimethoxybenzene was utilized in ether synthesis with boc-D-prolinol by reaction under basic conditions with sodium hydride in DMF. In certain cases, only the alcohol version of dimethoxybenzene was available, so the alcohol group on the reactant was first replaced with tosyl group with tosyl chloride and pyridine prior to introducing it into ether synthesis. (See, FIG. 3.) The final ether products were extracted with water and ethyl acetate, and then purified with flash chromatography with hexane and ethyl acetate solvents. The final ether products were N-deprotected and can be reacted with the same 4-chloro-2-(methylsulfonyl)pyrimidine and imidazole starting materials in the same manner as described above, to provide compounds 12-14 (FIG. 4).

### Example 11

NOS Enzyme Assays. Rat and human nNOS, murine macrophage iNOS, and bovine eNOS were recombinant enzymes, expressed in E. coli and purified as previously reported in the literature. To test for enzyme inhibition by a test compound, the hemoglobin capture assay was used to measure nitric oxide production. (See, Hevel, J. M. and Marietta, M.A. Nitric-oxide synthase assays. Methods Enzymol. 1994, 233, 250-258.) The assay was run at 37° C. in 100 mM HEPES buffer (10% glycerol; pH 7.4) in the presence of 10 μM L-arginine. The following were also included in the assay: 100 μM NADPH, 10 μM tetrahydrobiopterin, 1 mM CaCl₂, 11.6 μg/mL calmodulin and 3.0 μM oxyhemoglobin. For iNOS, calmodulin and CaCl₂ were omitted because iNOS is calcium independent; CaM is bound tightly. All NOS isozymes were used at a concentration of approximately 100 nM. The assay was run in a 96 well plate, using the Synergy 4 by BioTek, at the Northwestern University High Throughput Analysis Facility. The assay was run in triplicate; Forty-eight 100 μL reactions were performed at once. The addition of hemoglobin and NOS were automated with a maximum of a 30 second delay before the reactions could be recorded at 401 nm. The absorbance increase at 401 nm is due to the formation of NO via the conversion of oxyhemoglobin to methemoglobin. The IC₅₀ values were obtained using non-linear regression in GraphPad Prism5 software, and Kᵢ values were determined using the Cheng-Prusoff relationship Kᵢ=IC₅₀/(1+[S]/Kₘ), using the following known Kₘ values: rat nNOS 1.3 μM; murine iNOS 8.3 μM; bovine eNOS 1.7 μM.

### Example 12a

nNOS Dimerization Inhibition assay. Hek293 cells stably transduced to express rat nNOS were grown to confuency in DMEM supplemented with 10% FBS, pen/strep and 0.4 mg/mL G418. Cells were then plated into 96 well plates at 50,000 cells per well. After 12 h of growth a serial dilution of test compound was added with a vehicle control leaving one additional lane empty as a second activator control. Compounds and cells were allowed to interact for 48 h to allow for full degradation of undrugged active dimeric species. After 48 h lul of a 10 mM calcium ionophore stock was added. After 4 h, 50 uL of the media from each well was removed and mixed with 50 uL Griess Reagent 1 (Promega Griess assay kit). This mixture was incubated for 5 mins and then 50 uL Reagent 2 was added. The plate was read at 530 nm and an IC₅₀ calculated.

### Example 12b

eNOS Dimerization Inhibition assay. The nNOS protocol was followed substituting Hek293 cells stably transduced to express bovine eNOS

### Example 12c

iNOS Dimerization Inhibition assay. Raw 264.7 cells were grown to confuency in DMEM supplemented with 10% FBS and pen/strep. Cells were then plated into 96 well plates at 50,000 cells per well. After 12h of growth a serial dilution of test compound was added along with e.coli LPS (50 ug/ml final) and IFN-Y (500 ng/ml final) with a vehicle control. Compounds and cells were allowed to interact for 48 h to allow for full degradation of undrugged active dimeric species. After 48 h, 50 uL of the media from each well was removed and mixed with 50 uL Griess Reagent 1 (Promega Griess assay kit). This mixture was incubated for 5 mins and then 50 uL Reagent 2 was added. The plate was read at 530 nm and an IC₅₀ calculated.

As illustrated, the analogs contain a heme-binding group that enables selective targeting of the heme group in the oxygenase region of nNOS monomer. An aromatic structure, connected to the heme-binding group via an aliphatic carbon chain, interacts with surrounding residues which then produces an allosteric effect on the monomer. This allosteric effect prevents nNOS monomers to align and dimerize. In addition, the aliphatic carbon chain contains a β-turn configuration, allowing the aromatic structure to access the desired residues for improved nNOS binding. Analogs were assayed in vitro with enzyme, and validated for mechanism, potency, and selectivity against other NOS isoforms via crystallography and biological assays.

### Example 13

The protocol for the cell-based assay for HEK293 cells overexpressing rat nNOS is as follows and includes: (1) HEK293 cell culture and prep (2) Dosing with drug, cell lysis, and total protein analysis with BCA (3) Western Blot and image analysis to calculate IC50

HEK293 Cell Culture. Materials: media (DMEM+10% FBS), TryLE, PBS. 1. Warm media in 37C. Add 10 mL of media into 100 mm culture dish or T75 flask. 2. Thaw vial of cells in 37C bath until there is a sliver of ice (˜30 sec-1 min). 3. Pipette full vial of cells into the cell culture dish or flask with the media. 4. Incubate at 37C, 5% CO2 overnight. 5. Check cells the next day. If cells have adhered to plate, aspirate media off, and add fresh media. Return to incubator. If not, return cells to incubator and check later/change out fresh media. If cells are not doubling correctly, the vial maybe bad, will need to thaw a new vial.

HEK293 Cell Passage. 6. Pass cells every 2-3 days or when it has reached 80-100% confluency. 7. Aspirate off media. 8. Wash with 8 mL PBS. Aspirate off 9. Add 3-5 mL of TryLE to cells. Incubate at 37C for 5 mins. Check if cells have become suspended. May need to tap the sides with hand to get the cells to release. 10. Add 5-7 mL of media to neutralize TryLE. With serological pipette, resuspend cells fully, until there are no clumps. 11. Pass about 2-3 mL of suspended cells into fresh, warmed media in a new 100 mm dish. Media volume should be the balance to make up to 10mL total. 12. Return cells to incubator 37C, 5% CO2.

HEK293 Cell Seeding for Bioassay. Materials: Flat-bottom 96-well culture plate, TrypLE, DMEM+10%FBS. 13. Check cells for health (98-99% viability) and also confluency to see if there would be enough cells (In general, 80-100% confluent cells with 100 mm dish is more than enough for 2 plates of 96-well plate. 14. Follow HEK293 Cell Passage steps 7-10. 15. Once cells are suspended, count the cells to get concentration. Recommend to do two readings and take the average. 16. 200 uL liquid volume per well. Calculate the volume needed to fill the required number of 96-well plate. Also include a little bit extra volume in the calculation. 0.2 mL×(# of well+20 wells extra volume)=total liquid volume. 17. Seeding concentration should target 0.25×10∧6 viable cells/mL. Calculate the volume of cells required. Total liquid volume mL×0.25×10∧6 target viable cells/mL=Volume of Cells Average Cell Concentration. 18. Calculate total media volume to dilute cells. Total liquid volume−Volume of cells=Media volume. 19. In conical tube, dilute appropriate volume of cells with appropriate volume of media. Suspend gently but thoroughly, by flicking the tube or with serological pipette. 20. With multichannel pipette, aliquot 200 uL of dilute cells into each well. Be careful not to pipette up and down too much which can shear the cells. 21. Put plates of cells back in incubator at 37C at 5% CO2, and leave for 16-18 hours before adding drugs.

Drug Dosing. Materials: round-bottom 96-well plate for drugs, 0.1M HCl, Lysis buffer (M-per), Pierce BCA reagent plate. 22. Make 10 mM stocks of each drug, dissolved in 0.1M HCl. 23. On a 96-well plate, lay out the concentrations of drug that will be dosed in the corresponding cell wells. Column 1 is typically used as the “control”, containing just 0.1M HC1 without any drugs. Starting with Column 2, is 10 mM concentration of drug, and then the rest of the columns are serially diluated, 1:3 to generate a concentration range from 100 uM->5 nM. 24. Using multi-channel pipette, add 2 uL of drug in each well into its corresponding well of cells. 25. Swirl contents to mix gently. 26. Return cells to incubator at 37C, 5% CO2 for 60-72 hours. Check each day for cell health, and confluency. 27. Harvest cells: aspirate media, wash cells with PBS and aspirate. 28. Add lysis buffer (RIPA and M-per both work). About 40 uL per well. 29. Let plate shake for 5 minutes at room temperature. 30. Check total protein content in cell lysis with BCA assay. Use 25uL total volume of diluted sample (usually by 2×) in 200 uL volume of working reagent for assay. Follow Pierce protocol to prepare working reagents. Aliquot reagents and mix into samples quickly by pipetting up and down, and incubate on shaker for 30 mins at 37C. Read at OD540. Typical readings are usually 0.65-0.85. Note: there is a program on UV/Spec instrument that has the 30 mins shaking programmed into the protocol before reading OD540. 31. Lysis samples need to be stored at −80C or liquid nitrogen if not immediately being used for assay.

Quantitative Western Blot. 32. Choose which samples to load, and normalize loading volume if a sample is outside of standard deviation. 33. Load 15-20 uL with 4× LDS loading buffer per lane, onto NUPAGE 4-12% Bis-Tris gels. If BCA assay gave results in lower OD range, then load closer to 20 uL volume. Load about 5 uL of Protein standard on the outer lanes (2 lanes total) to help with cutting the gel later and determine when SDS-PAGE is done. 34. Run SDS-PAGE in MOPS buffer at 4C, 200 v, 65-70 minutes on ice in the cold room. Can stop run when bottom band runs to about more than ¾ down the gel. 35. Cut gel at around 75-80 kDa band. Transfer top gel piece (with the higher molecular weight proteins) to membrane for blotting. We used iBlot dry transfer (8 minutes, 20-25 v), wet transfer (1-2 hours, 400 amps). 36. Make about 1 L of TBST=1% Tween+TBS. 37. Incubated membrane in blocking buffer on shaker for 1 hour room temperature, or 4C overnight. One membrane can use about 50 mL volume. Blocking buffer=50 mL of TBST+2.5 g of fat-free powder milk. 38. Discard blocking buffer. Wash membrane 3 times for 5 minutes each with TBST. 39. Add primary nNOS antibody to membrane (from Cell Signal C707, with TBST+5% BSA). Incubate on shaker overnight at 4C in cold room. 40. Save primary nNOS antibody for future use at 20C. Wash membrane 3 times for 5 minutes each with TBST. 41. Add secondary antibody to membrane, which was made with 50mL TBST +5uL of ECL anti-rabbit IgG horseradish peroxidase (HRP), or whatever secondary antibody you prefer. Incubate on shaker at room temperature for 1 hour. 42. Discard secondary antibody. Wash 3 times for 5 minutes each TBST. 43. Membrane is now ready to be imaged. Lay the membrane on platform. Add a few mL of HRP to the membrane. Let image develop for 30 seconds or so. Use instructions in the imaging program to take a chemiluminescence high-resolution image. Best results are with images that have little to no background.

Image Analysis and IC50 Calculation. Use image software to identify lanes and select for dimer and monomer nNOS bands. It will read the intensity of the peaks as fractions: Fraction of dimers=Intensity of dimer peak/(Total of dimer and monomer peak combined). 45. Record fraction of dimers. May need to subtract out the background intensity. Graph values against drug concentration, and determine IC50 (can also use fitting function, IC50 calculation software, etc).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A compound or a salt thereof having a formula: 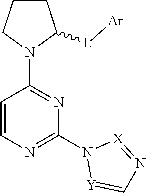
wherein:
X and Y are selected from CH and N with the proviso that both of X and Y are not N;
L is a divalent alkylene moiety optionally substituted with substituents selected from —O—, —C(O)NH—, and —NHC(O)—; and
Ar is selected from aryl, heteroaryl and substituted aryl and heteroaryl moieties, such substituents selected from alkyl and alkoxy substituents.

2. The compound of claim 1, wherein both of X and Y are CH.

3. The compound of claim 1, wherein L has a formula —(CH₂)ₘ—Z—(CH₂)ₙ—; m is selected from 0-4, n is selected from 0-4, and Z is selected from —CH₂—, —O—, —C(O)NH—, and —NHC(O)—.

4. The compound of claim 1, wherein Ar is a saturated or unsaturated carbon homocycle or heterocycle moiety comprising one 5- or 6-membered ring or comprising two or three fused 5- or 6-membered rings optionally substituted with alkyl or alkyoxy substituents.

5. The compound of claim 1, wherein Ar is selected from phenyl, 1,3-benzodioxole, indole, benzimidazole, pyridine, pyrimidine, and pyridazine.

6. The compound of claim 1 having a formula selected from:

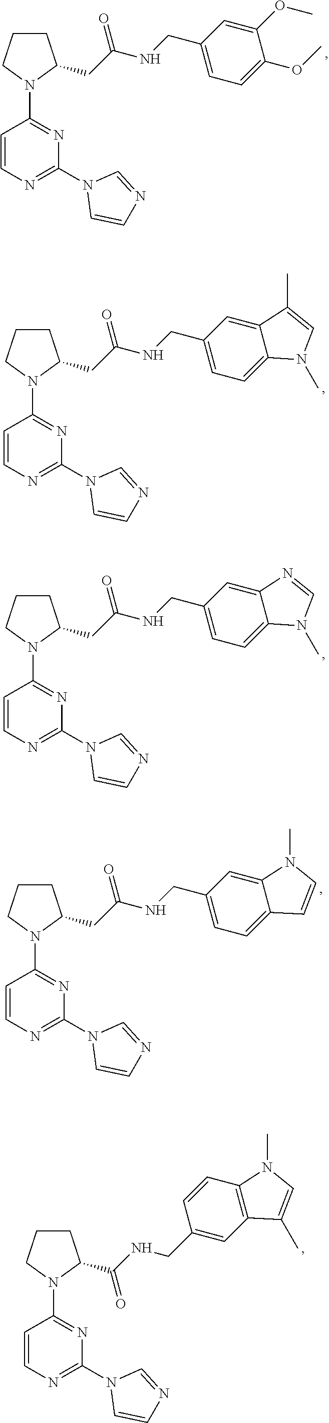 [SMILES: COc1ccc(CNC(=O)C[C@H]2CCCN2c2ccnc(-n3ccnc3)n2)cc1OC ; Cc1cn(C)c2ccc(CNC(=O)C[C@H]3CCCN3c3ccnc(-n4ccnc4)n3)cc12 ; Cn1cnc2cc(CNC(=O)C[C@H]3CCCN3c3ccnc(-n4ccnc4)n3)ccc21 ; Cn1ccc2ccc(CNC(=O)C[C@H]3CCCN3c3ccnc(-n4ccnc4)n3)cc21 ; Cc1cn(C)c2ccc(CNC(=O)[C@H]3CCCN3c3ccnc(-n4ccnc4)n3)cc12]

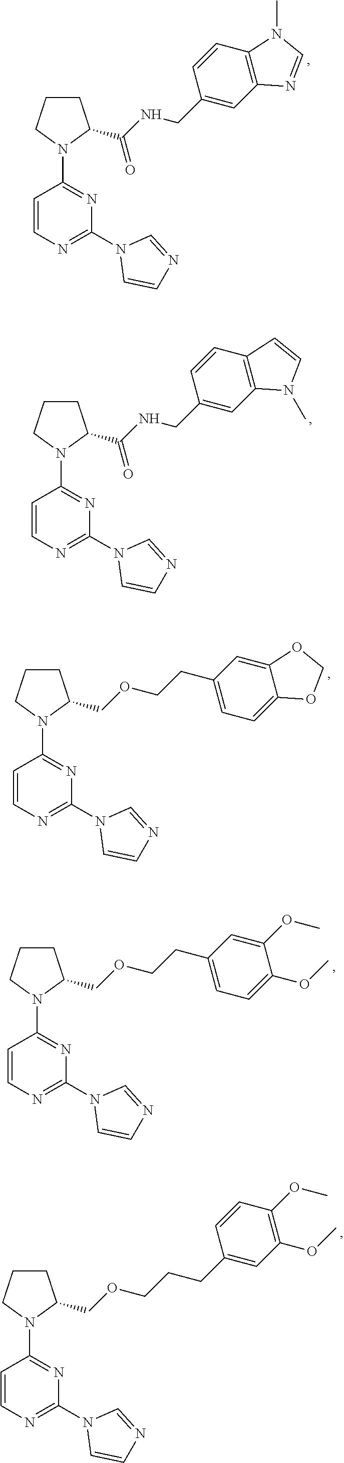 [SMILES: Cn1cnc2cc(CNC(=O)[C@H]3CCCN3c3ccnc(-n4ccnc4)n3)ccc21 ; Cn1ccc2ccc(CNC(=O)[C@H]3CCCN3c3ccnc(-n4ccnc4)n3)cc21 ; c1cn(-c2nccc(N3CCC[C@@H]3COCCc3ccc4c(c3)OCO4)n2)cn1 ; COc1ccc(CCOC[C@H]2CCCN2c2ccnc(-n3ccnc3)n2)cc1OC ; COc1ccc(CCCOC[C@H]2CCCN2c2ccnc(-n3ccnc3)n2)cc1OC]

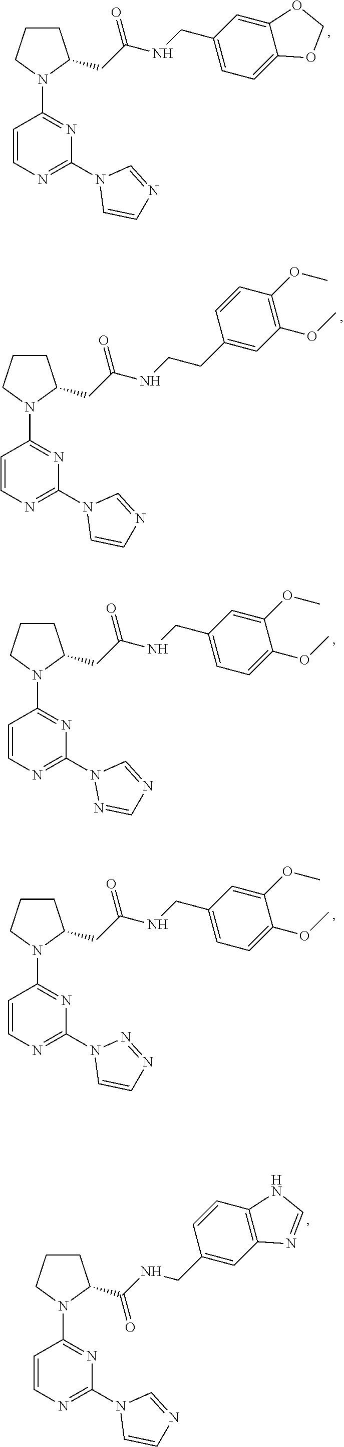 [SMILES: O=C(C[C@H]1CCCN1c1ccnc(-n2ccnc2)n1)NCc1ccc2c(c1)OCO2 ; COc1ccc(CCNC(=O)C[C@H]2CCCN2c2ccnc(-n3ccnc3)n2)cc1OC ; COc1ccc(CNC(=O)C[C@H]2CCCN2c2ccnc(-n3cncn3)n2)cc1OC ; COc1ccc(CNC(=O)C[C@H]2CCCN2c2ccnc(-n3ccnn3)n2)cc1OC ; O=C(NCc1ccc2[nH]cnc2c1)[C@H]1CCCN1c1ccnc(-n2ccnc2)n1]

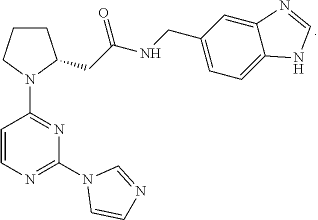 [SMILES: O=C(C[C@H]1CCCN1c1ccnc(-n2ccnc2)n1)NCc1ccc2[nH]cnc2c1]

7. A pharmaceutical composition comprising the compound of claim 1 and a suitable carrier.

8. A method of treating a disease or disorder associated with neuronal nitric oxide synthase activity in a subject in need thereof, the method comprising administering to the subject the compound of claim 1.

9. The method of claim 8, wherein the disease or disorder is neuropathic pain associated with neuronal nitric oxide synthase activity.

10. The method of claim 8, wherein the disease or disorder is a neurodegenerative disease.

11. The method of claim 10, wherein the neurodegenerative disease is Alzheimer's disease.

12. The method of to claim 10, wherein the neurodegenerative disease is Parkinson's disease.
